(19) United States
(12) Patent Application Publication
(10) Pub. No.: US 2011/0305678 A1
(43) Pub. Date: Dec. 15, 2011

(54) USE OF MESENCHYMAL STEM CELLS FOR TREATING GENETIC DISEASES AND DISORDERS

(76) Inventors: Timothy Varney, Baltimore, MD (US); Charles Randal Mills, Finksburg, MD (US); Alla Danilkovitch, Columbia, MD (US)

(21) Appl. No.: 13/216,880

(22) Filed: Aug. 24, 2011

Related U.S. Application Data
(63) Continuation of application No. 12/581,347, filed on Oct. 19, 2009, now abandoned.
Application No. 12/581,347 is a continuation of application No. 11/651,878, filed on Jan. 10, 2007, now abandoned.
(60) Provisional application No. 60/758,387, filed on Jan. 12, 2006.

(51) Int. Cl. A61K 35/12; A61P 11/00; A61K 48/00

(52) U.S. Cl. 424/93.7

(57) ABSTRACT

A method of treating a genetic disease or disorder such as, for example, cystic fibrosis, Wilson's disease, amyotrophic lateral sclerosis, or polycystic kidney disease, in an animal comprising administering to said animal mesenchymal stem cells in an amount effective to treat the genetic disease or disorder in the animal.

### RELATED APPLICATIONS

This application claims priority to, and is a continuation of, U.S. application Ser. No. 12/581,347, filed on Oct. 19, 2009, which claims priority to, and is a continuation of, U.S. application Ser. No. 11/651,878 having a filing date of Jan. 10, 2007, which are incorporated herein by reference in their entirety, and which claims priority to U.S. Provisional Patent Application No. 60/758,387, filed Jan. 12, 2006, which is also incorporated herein by reference in its entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

[Not Applicable]

### BRIEF SUMMARY OF THE INVENTION

The present technology relates to mesenchymal stem cells. More particularly, the present technology relates to the use of mesenchymal stem cells for treating genetic diseases and disorders. Still more particularly, the present technology relates to the use of mesenchymal stem cells for treating genetic diseases or disorders that are characterized by inflammation of at least one tissue and/or at least one organ.

### BRIEF DESCRIPTION OF SEVERAL VIEWS OF THE DRAWINGS

[Not Applicable]

### DETAILED DESCRIPTION OF THE INVENTION

Mesenchymal stem cells (MSCs) are multipotent stem cells that can differentiate readily into lineages including osteoblasts, myocytes, chondrocytes, and adipocytes (Pittenger, et al., Science, Vol. 284, pg. 143 (1999); Haynesworth, et at., Bone, Vol. 13, pg. 69 (1992); Prockop, Science, Vol. 276, pg. 71 (1997)). In vitro studies have demonstrated the capability of MSCs to differentiate into muscle (Wakitani, et at., Muscle Nerve, Vol. 18, pg. 1417 (1995)), neuronal-like precursors (Woodbury, et al., J. Neurosci. Res., Vol. 69, pg. 908 (2002); Sanchez-Ramos, et at., Exp. Neurol., Vol. 171, pg. 109 (2001)), cardiomyocytes (Toma, et al., Circulation, Vol. 105, pg. 93 (2002); Fakuda, Artif. Organs, Vol. 25, pg. 187 (2001)) and possibly other cell types. In addition, MSCs have been shown to provide effective feeder layers for expansion of hematopoietic stem cells (Eaves, et al., Ann. N.Y. Acad. Sci., Vol. 938, pg. 63 (2001); Wagers, et at., Gene Therapy, Vol. 9, pg. 606 (2002)). Recent studies with a variety of animal models have shown that MSCs may be useful in the repair or regeneration/of damaged bone, cartilage, meniscus or myocardial tissues (DeKok, et al., Clin. Oral Implants Res., Vol. 14, pg. 481 (2003)); Wu, et al., Transplantation, Vol. 75, pg. 679 (2003); Noel, et al., Curr. Opin. Investig. Drugs, Vol. 3, pg. 1000 (2002); Ballas, et al., J. Cell. Biochem. Suppl., Vol. 38, pg. 20 (2002); Mackenzie, et at., Blood Cells Mol. Dis., Vol. 27 (2002)). Several investigators have used MSCs with encouraging results for transplantation in animal disease models including osteogenesis-imperfecta (Pereira, et al., Proc. Nat. Acad. Sci., Vol. 95, pg. 1142 (1998)), parkinsonism (Schwartz, et al., Hum. Gene Ther., Vol. 10, pg. 2539 (1999)), spinal cord injury (Chopp, et al., Neuroreport, Vol. 11, pg. 3001 (2000); Wu, et al., J. Neurosci. Res., Vol. 72, pg. 393 (2003)) and cardiac disorders (Tomita, et al., Circulation, Vol. 100, pg. 247 (1999); Shake, et al., Ann. Thorac. Surg., Vol. 73, pg. 1919 (2002)). Importantly, promising results also have been reported in clinical trials for osteogenesis imperfecta (Horwitz, et al., Blood, Vol. 97, pg. 1227 (2001); Horowitz, et al. Proc. Nat. Acad. Sci., Vol. 99, pg. 8932 (2002)) and enhanced engraftment of heterologous bone marrow transplants (Frassoni, et al., Int. Society for Cell Therapy, SA006 (abstract) (2002); Koc, et al., J. Clin. Oncol., Vol. 18, pg. 307 (2000)).

It has been surprisingly discovered that mesenchymal stem cells, when administered systemically, such as by intravenous or intraosseous administration, migrate toward and engraft within inflamed tissue. Thus, in accordance with an aspect of the present technology, there is provided a method of treating a genetic disease or disorder in an animal, and more particularly a method of treating a genetic disease or disorder that is characterized by at least one of an inflamed tissue or organ of the animal. The method comprises administering to the animal mesenchymal stem cells in an amount effective to treat the genetic disease or disorder in the animal.

Although the scope of the present technology is not to be limited to any theoretical reasoning, infused mesenchymal stem cells (MSCs) home to, i.e., migrate toward, and engraft within inflamed tissue. Inflammatory involvement has been described for several genetic diseases including, but not limited to, polycystic kidney disease, cystic fibrosis, Wilson's Disease, Gaucher's Disease, and Huntington's Disease, for example. The presence of inflammation within the tissue or organs affected by these and other genetic disorders may facilitate homing of the MSCs to the inflamed tissues and/or organs, and facilitate engraftment of the MSCs.

The administration of the MSCs may correct tissue and/or organ dysfunction caused by a genetic defect in that the MSCs carry a wild-type copy of the gene that is defective in the animal being treated. The administration of the MSCs to the patient results in the engraftment of cells that carry the wild-type gene to tissues and/or organs affected by the disease. The engrafted MSCs will differentiate according to the local environment. Upon differentiation, the MSCs will express the wild-type version of the protein that is defective or absent from the surrounding tissue. Engraftment and differentiation of the donor MSCs within the defective tissue and/or organ will correct the tissue and/or organ function.

In addition, genetic transduction of the donor MSCs is not required in that the donor MSCs have an endogenous wild-type version of the same gene that is defective in the animal being treated. The correction of tissue and/or organ function results from the presence of such wild-type gene.

The use of MSCs as a vehicle for wild-type gene delivery will provide normal copies of all genes which, when mutated, lead to the development of the genetic disease to be treated. This is true (1) whether the gene defect(s) has (have) been identified, (2) whether the contribution of the mutated form of the gene(s) to the development of the disease is known, and (3) whether the disease results from a single genetic mutation or a combination of genetic mutations. The expression of the normal form of the proteins which, when non-functional, contribute to the development of the disease, will improve or correct the function of tissues impaired by the disease.

In general, the genetic disease or disorder to be treated is a genetic disease or disorder characterized by at least one inflamed tissue or organ, although other genetic diseases and disorders may be treated as well. Genetic diseases or disorders which may be treated in accordance with the present technology include, but are not limited to, cystic fibrosis, polycystic kidney disease, Wilson's disease, amyotrophic lateral sclerosis (or ALS or Lou Gehrig's Disease), Duchenne muscular dystrophy, Becker muscular dystrophy, Gaucher's disease, Parkinson's disease, Alzheimer's disease, Huntington's disease, Charcot-Marie-Tooth syndrome, Zellweger syndrome, autoimmune polyglandular syndrome, Marfan's syndrome, Werner syndrome, adrenoleukodystrophy (or ALD), Menkes syndrome, malignant infantile osteopetrosis, spinocerebellar ataxia, spinal muscular atrophy (or SMA), and glucose galactose malabsorption.

For example, cystic fibrosis (CF) is a genetic disorder characterized by impaired functionality of secretory cells in the lungs, pancreas and other organs. The secretion defect in these cells is caused by the lack of a functional copy of the Cystic Fibrosis Transmembrane Conductance Regulator (CFTR) gene. Mutations in the CFTR gene result in the appearance of an abnormally thick, sticky mucus lining in the lungs that clogs air passages and leads to life-threatening infections. Also, thick secretions in the pancreas prevent digestive enzymes from reaching the intestines, leading to poor weight gain.

MSC administration may be employed to treat CF symptoms by providing wild type (normal) CFTR genes to tissues affected by the disease. The localization of systemically delivered MSCs to the lungs is effected by both the path of circulatory flow and by the migration response of MSCs to inflamed tissues. CF patients typically suffer from frequent Pseudomonas aeruginosa infections of the lungs. Successive rounds of Pseudomonas infection and resolution are accompanied by inflammation and scarring. Inflammatory markers in the lungs of CF patients include TNF-α and MCP-1, chemokines that are known to promote MSC recruitment.

Following integration within affected tissues, the MSCs differentiate (mature) according to the local environment and begin producing functionally normal CFTR protein. The presence of cells containing an active form of the protein improves or corrects the secretory impairment observed in CF tissues. MSC delivery also limits the progression of fibrosis and scar expansion in the lungs of CF patients.

Wilson's disease is a genetic disorder of copper transport, resulting in copper accumulation and toxicity in the liver, brain, eyes and other sites. The liver of a person who has Wilson's disease does not release copper into the bile correctly. A defect in the ATP7B gene is responsible for the symptoms of Wilson's disease.

Copper accumulation in the liver results in tissue damage characterized by inflammation and fibrosis. The inflammatory response of Wilson's disease involves TNF-α, a chemokine known to promote the recruitment of MSCs to damaged tissue. Systemically delivered MSCs therefore migrate to regions of inflamed liver in Wilson's disease patients. Upon engraftment, the MSC differentiate to form hepatocytes and initiate expression of the normal copy of the ATP7B gene and production of functional ATP7B protein.

Hepatocytes derived from exogenously delivered MSCs therefore will carry out normal copper transport, thereby reducing or ameliorating excess copper accumulation in the liver. Location-specific maturation of MSCs may reduce the buildup of copper in the brain and eyes as well. The reduction of copper accumulation in these tissues will resolve the symptoms of Wilson's disease in patients treated by MSC therapy.

Amyotrophic lateral sclerosis (ALS or Lou Gehrig's Disease) is a neurological disorder characterized by progressive degeneration of motor neuron cells in the spinal cord and brain, which results ultimately in paralysis and death. The SOD1 gene (or ALS 1 gene) is associated with many cases of familial ALS (Nature, Vol. 362:59-62). The enzyme coded for by SOD1 removes superoxide radicals by converting them into non-harmful substances. Defects in the action of SOD1 result in cell death due to excess levels of superoxide radicals. Several different mutations in this enzyme all result in ALS, making the exact molecular cause of the disease difficult to ascertain. Other known genes that, when mutated, contribute to the onset of ALS include ALS2 (Nature Genetics, 29 (2):166-73.), ALS3 (Am J Hum Genet, 2002 January; 70 (1):251-6.) and ALS4 (Am J Hum Genet. June; 74 (6)).

It is suspected that there are several currently unidentified genes that contribute to susceptibility to ALS. This is particularly the case in patients with non-familial ALS. MSC treatment provides normal copies of these genes to ALS patients because donor MSCs are obtained from healthy donors and mutations that result in the development of ALS are rare.

The use of MSCs as a vehicle for wild type gene delivery will provide normal copies of all genes which, when mutated, lead to the development of ALS. This is true (1) whether the gene defect(s) has been identified, (2) whether the contribution of the mutated form of the gene(s) to the development of ALS is known, and (3) whether the disease results from a single genetic mutation or a combination of genetic mutations. The expression of the normal form of the proteins which, when non-functional, contribute to the development of ALS will restore muscle function in ALS patients.

Muscular dystrophies are diseases involving progressive wasting of the voluntary muscles, eventually affecting the muscles controlling pulmonary function. Duchenne and Becker muscular dystrophies are both caused by mutations in the gene that encodes the protein dystrophin. In Duchenne's muscular dystrophy, the more severe disease, normal dystrophin protein is absent. In the milder Becker's muscular dystrophy, some normal dystrophin is made, but in insufficient amounts.

Dystrophin imparts structural integrity to muscle cells by connecting the internal cytoskeleton to the plasma membrane. Muscle cells lacking or having insufficient amounts of dystrophin also are relatively permeable. Extracellular components can enter these more permeable cells, this increasing the internal pressure until the muscle cell ruptures and dies. The subsequent inflammatory response can add to the damage. The inflammatory mediators in muscular dystrophy include TNF-α (Acta Neuropathol (Berl)., 2005 February; 109 (2):217-25. Epub 2004 Nov. 16), a chemokine known to promote MSC migration to damaged tissue.

Delivery of MSCs containing a normal dystrophin gene treats the symptoms of Duchenne's and Becker's muscular dystrophy in the following manner. MSC migration to degenerative muscle will result in MSC differentiation according to the local environment, in this case to form muscle cells. MSCs that differentiate to form muscle will express normal dystrophin protein, because these cells carry the normal dystrophin gene. MSC-derived muscle cells will fuse with endogenous muscle cells, providing normal dystrophin protein to the multinucleated cell. The successful fusion of dystrophin-expressing MSCs with differentiating human myoblasts has been reported in an article entitled, “Human mesenchymal stem cells ectopically expressing full-length dystrophin can complement Duchenne muscular dystrophy myotubes by cell fusion.” (Goncalves, et al., Advance Access published online on Dec. 1, 2005 in Human Molecular Genetics.) The greater the degree of MSC engraftment within degenerative muscle, the closer the muscle tissue will resemble normal muscle structurally and functionally.

Gaucher's disease results from the inability to produce the enzyme glucocerebrosidase, a protein that normally breaks down a particular kind of fat called glucocerebroside. In Gaucher's disease, glucocerebroside accumulates in the liver, spleen, and bone marrow.

Gaucher's disease may be treated by the delivery of MSCs that harbor a normal copy of the gene that encodes glucocerebrosidase. Tissue damage caused by glucocerebroside accumulation produces an inflammatory response that causes the migration of MSCs to damaged regions. The inflammatory response in Gaucher's disease involves TNF-α, a cytokine known to recruit MSCs to areas of tissue damage (Eur Cytokine Netw., 1999 June; 10 (2):205-10). Once engrafted within damaged tissue, MSCs will differentiate to replace missing cell types according to local environmental cues. MSC derived cells will have the ability to break down glucocerebroside normally, due to the expression of ability to express active glucocerebrosidase by such cells.

Intravenously delivered glucocerebrosidase enzyme is effective in slowing the progression of, or even reversing the symptoms of Gaucher's disease (Biochem Biophys Res Commun., 2004 May 28; 318 (2):381-90.). It is not known if wild type MSCs will produce glucocerebrosidase that will be available externally to the MSC-derived cell that produces the enzyme. If so, glucocerebrosidase expression by exogenously derived MSCs will reduce glucocerebroside levels in surrounding tissue. The benefit of MSC therapy for Gaucher's disease in this case would lie not only in the contribution of cells that have the ability to break down glucocerebroside, but also in the fact that these cells can provide glucocerebrosidase to neighboring cells as well, resulting in the reduction of glucocerebroside in native tissue.

Parkinson's disease (PD) is a motor system disorder that results from the loss of dopamine-producing brain cells. The primary symptoms of PD are tremor, stiffness of the limbs and trunk, bradykinesia, and impaired balance and coordination. A classic pathological feature of the disease is the presence of an inclusion body, called the Lewy body, in many regions of the brain.

It is believed generally that there is a genetic component to PD, and that a variety of distinct mutations may result in disease onset. One gene thought to be involved in at least some cases of Parkinson's is ASYN, which encodes the protein alpha-synuclein. The accumulation of alpha-synuclein in Lewy body plaques is a feature of both Parkinson's and Alzheimer's diseases.

It is not yet clear whether alpha-synuclein accumulation is a root cause of neural damage in Parkinson's or a result of neural cell death. If alpha-synuclein buildup is a primary cause of neural degeneration, then one possibility is that one or more additional proteins responsible for regulating the expression or accumulation of alpha synuclein damage has declined with age. One mechanism by which MSC therapy may treat PD therefore, is through providing a renewed source of one or more of such regulatory proteins.

Regardless of the genetic basis of the disease, delivery of MSCs to PD patients results in the replacement of dopamine-producing cells. Inflammation resulting from neuronal cell death should cause MSC migration directly to affected regions of the brain.

Alzheimer's disease results in a progressive loss in the ability to remember facts and events, and eventually to recognize friends and family. The pathology in the brains of Alzheimer's patients is characterized by the formation of lesions made of fragmented brain cells surrounded by amyloid-family proteins.

Delivery of MSCs that contain normal copies of the presenilin-1 (PS1), presenilin-2 (PS2) and possibly other, as yet unidentified, genes will treat the complications of Alzheimer's disease. The inflammation resulting from brain cell fragmentation that is characteristic of the disease attracts MSCs to migrate into the area. MSCs can differentiate into neural cell types when located within damaged neural tissue. Further, the metalloproteinases expressed and secreted by MSCs reduces the characteristic lesions found in the brains of Alzheimer's patients by degrading amyloid proteins and other protein types within these plaques. Resolution of amyloid plaques will provide an opportunity for the differentiation of MSCs and endogenous stem cells to form neurons.

Huntington's disease (HD) is an inherited, degenerative neurological disease that leads to decreased control of movement, loss of intellectual faculties and emotional disturbance. A mutation in the HD gene, the gene that encodes the Huntington protein, eventually results in nerve degeneration in the basal ganglia and cerebral cortex of the brain.

How mutations in the HD gene result in Huntington's disease is not clear. The inflammation associated with neural degeneration, however, provides an environment that is conducive to MSC recruitment. MSC engraftment to these regions will lead to differentiation according to the local environment, including MSC maturation to form neurons that carry a normal form of the HD gene. One effect of MSC therapy, therefore, is to replace neurons lost to neural degeneration.

Contributing factors to the onset and/or progression of Huntington's disease may include an age-related decrease in regulatory proteins that control the production level of Huntington protein. Thus, the administration of MSCs restores the availability of such regulatory constituents.

Charcot-Marie-Tooth syndrome (CMT) is characterized by a slow progressive degeneration of the muscles in the foot, lower leg, hand, and forearm and a mild loss of sensation in the limbs, fingers, and toes.

The genes that produce CMT when mutated are expressed in Schwann cells and neurons. Several different and distinct mutations, or combinations of mutations, can produce the symptoms of CMT. Different patterns of inheritance of CMT mutations are also known. One of the most common forms of CMT is Type 1A. The gene that is mutated in Type 1A CMT is thought to encode the protein PMP22, which is involved in coating peripheral nerves with myelin, a fatty sheath that is important in nerve conductance. Other types of CMT include Type 1B, autosomal-recessive, and X-linked.

Delivery of MSCs expressing a normal copy of the Type 1A CMT gene, Type 1B CMT gene and/or other genes may restore the myelin coating of peripheral nerves. A component of the inflammatory response in degenerative regions involves the production and secretion of MCP-1 (monocyte chemoattractant protein (J. Neurosci Res., 2005 Sep. 15; 81 (6):857-64), a cytokine known to support the homing of MSCs to damaged tissue. The mechanism of restoring the structure and functionality of degenerative tissue will depend on the particular mutation involved in promoting the disease.

Other genetic diseases that may be treated by administering MSCs are listed below.

Polycystic kidney disease: Delivery of a normal form of the PKD1 gene by may inhibit cyst formation.

Zellweqer syndrome: Delivery of a normal copy of the PXR1 gene by the MSCs corrects peroxisome function, imparting normal cellular lipid metabolism and metabolic oxidation.

Autoimmune polyglandular syndrome: The disease will be treated by delivery of MSCs expressing a normal copy of the AIRE (autoimmune regulator) gene and/or regeneration of glandular tissue destroyed during disease progression.

Marfan's syndrome: Delivery of MSCs expressing a normal form of the FBN1 gene will result in the production of fibrillin protein. The presence of fibrillin will impart normal structural integrity to connective tissues.

Werner syndrome: Delivery of MSCs expressing normal form of the WRN gene provides a source of cells for tissue turnover that do not age prematurely.

Adrenoleukodystrophy (ALD): Delivery of MSCs expressing a normal form of the ALD gene results in correct neuron myelination in the brain and/or will lead to regeneration of damaged areas of the adrenal gland.

Menkes syndrome: Delivery of MSCs that express a normal copy of an as yet unidentified gene or genes on the X chromosome that have the capability of absorbing copper will resolve disease symptoms.

Malignant infantile osteopetrosis: MSCs carry normal copies of genes that, when mutated, contribute to the onset of malignant infantile osteopetrosis. These genes include the chloride channel 7 gene (CLCN7), the osteopetrosis associated transmembrane protein (OSTM1) gene, and the T-cell immune regulatory (TCIRG1) gene. MSC delivery may correct the osteoblast/osteoclast ratio by providing MSCs that may act as osteoblast precursors and/or precursors to other cell types that control osteoclast differentiation.

Spinocerebellar ataxia: Delivery of MSCs that express a normal form of the SCA1 gene provides cells that will differentiate to form new neurons that produce the ataxin-1 protein (the product of the SCA1 gene) at appropriate levels to replace host neurons lost to neural degeneration. It is also possible that MSC engraftment will provide proteins that regulate the expression of the ataxin-1 protein.

Spinal muscular atrophy: Delivery of MSCs that express a normal copy of the SMA gene will provide cells that will differentiate to form new motor neurons to replace neurons that have died during disease progression.

Glucose galactose malabsorption: Delivery of MSCs expressing normal copies of the SGLT1 gene will correct glucose and galactose transport across the intestinal lining.

It is to be understood, however, that the scope of the present technology is not to be limited to the treatment of any particular genetic disease or disorder.

In one embodiment, the animal to which the mesenchymal stem cells are administered is a mammal. The mammal may be a primate, including human and non-human primates.

In general, the mesenchymal stem cell (MSC) therapy is based, for example, on the following sequence: harvest of MSC-containing tissue, isolation and expansion of MSCs, and administration of the MSCs to the animal, with or without biochemical manipulation.

The mesenchymal stem cells that are administered may be a homogeneous composition or may be a mixed cell population enriched in MSCs. Homogeneous mesenchymal stem cell compositions may be obtained by culturing adherent marrow or periosteal cells, and the mesenchymal stem cells may be identified by specific cell surface markers which are identified with unique monoclonal antibodies. A method for obtaining a cell population enriched in mesenchymal stem cells is described, for example, in U.S. Pat. No. 5,486,359. Alternative sources for mesenchymal stem cells include, but are not limited to, blood, skin, cord blood, muscle, fat, bone, and perichondrium.

Compositions having greater than about 95%, usually greater than about 98%, of human mesenchymal stem cells can be achieved using techniques for isolation, purification, and culture expansion of mesenchymal stem cells. For example, isolated, cultured mesenchymal stem cells may comprise a single phenotypic population (about 95% or about 98% homogeneous) by flow cytometric analysis of expressed surface antigens. The desired cells in such composition are identified as expressing a cell surface marker (e.g., CD73, CD105, or CD166) specifically bound by an antibody produced from hybridoma cell line SH2, ATCC accession number HB 10743, an antibody produced from hybridoma cell line SH3, ATCC accession number HB 10744, or an antibody produced from hybridoma cell line SH4, ATCC accession number HB 10745.

The mesenchymal stem cells may be administered by a variety of procedures. For example, the mesenchymal stem cells may be administered systemically, such as by intravenous, intraarterial, intraperitoneal, or intraosseous administration. The MSCs also may be delivered by direct injection to tissues and organs affected by the disease. In one embodiment, the mesenchymal stem cells are administered intravenously. In another embodiment, the mesenchymal stem cells are administered intraosseously.

The mesenchymal stem cells may be from a spectrum of sources, including allogeneic, autologous, and xenogeneic.

In one embodiment, prior to the administration of the donor mesenchymal stem cells, the host mesenchymal stem cell population is reduced, which increases donor MSC persistence. Reduction of the host mesenchymal stem cell population may be reduced by any of a variety of means known to those skilled in the art, including, but not limited to, partial or full body irradiation, and/or chemoablative or nonablative procedures.

In one non-limiting embodiment, the host is subjected to partial or full body irradiation prior to administration of the donor MSCs. The radiation may be administered as a single dose, or in multiple doses. In one embodiment, the radiation is administered in a total amount of from about 8 Grays (Gy) to about 12 Grays (Gy). In another embodiment, the radiation is administered in a total amount of from about 10 Gy to about 12 Gy. The amount of radiation to be administered and the number of doses administered are dependent upon a variety of factors, including the age, weight, and sex of the patient, and the general health of the patient at the time of administration.

In a non-limiting embodiment, when the host MSC population is reduced through partial or full body irradiation and/or chemoablative or nonablative procedures, bone marrow cells are administered along with the MSCs in order to reconstruct the host's hematopoietic system. The bone marrow cells may be administered systemically by methods such as, for example, intravenous, intraarterial, intraperitoneal, or intraosseous administration. The amount of bone marrow cells to be administered is dependent on a variety of factors, including the age, weight, and sex of the patient, the radiation and/or chemoablative or nonablative treatment given to the patient, the general health of the patient, and the source of the bone marrow.

In one embodiment, the bone marrow cells are autologous to the patient. In a further embodiment, the autologous bone marrow cells are administered in an amount of from 1×10⁷ cells to about 1×10⁸ cells per kg of body weight.

In another embodiment, the bone marrow cells are allogeneic to the patient. In a further embodiment, the allogeneic bone marrow cells are administered in an amount of from about 1×10⁸ cells to about 3×10⁸ cells per kg of body weight.

The mesenchymal stem cells are administered in an amount effective to treat the genetic disease or disorder in an animal. In one embodiment, the mesenchymal stem cells are administered in an amount of from about 1×10⁶ MSCs per kilogram (kg) of body weight to about 10×10⁶ MSCs per kg of body weight. In another embodiment, the mesenchymal stem cells are administered in an amount of from about 4×10⁶ MSCs per kg of body weight to about 8×10⁶ MSCs per kg of body weight. In yet another embodiment, the mesenchymal stem cells are administered in an amount of about 8×10⁶ MSCs per kg of body weight. The mesenchymal stem cells may be administered once, or the mesenchymal stem cells may be administered two or more times at periodic intervals of from about 3 days to about 7 days, or the mesenchymal stem cells may be administered chronically, i.e., during the entire lifetime of the animal, at periodic intervals of from about 1 month to about 12 months. The amount of mesenchymal stem cells to be administered and the frequency of administration are dependent upon a variety of factors, including the age, weight, and sex of the patient, the genetic disease or disorder to be treated, and the extent and severity thereof.

The mesenchymal stem cells may be administered in conjunction with an acceptable pharmaceutical carrier. For example, the mesenchymal stem cells may be administered as a cell suspension in a pharmaceutically acceptable liquid medium for injection. In one embodiment, the pharmaceutically acceptable liquid medium is a saline solution. The saline solution may contain additional materials such as dimethylsufoxide (DMSO) and human serum albumin.

The present technology provides a method of treating a genetic disease or disorder in an animal (including a human) wherein said genetic disease or disorder is characterized by at least one of an inflamed tissue or organ of said animal, said method comprising administering to said animal mesenchymal stem cells in an amount effective to treat said genetic disease or disorder in said animal. The genetic disease or disorder can be, for example, cystic fibrosis, polycystic kidney disease, Wilson's disease, amyotrophic lateral sclerosis, Duchenne muscular dystrophy, Becker muscular dystrophy, Gaucher's disease, Parkinson's disease, Alzheimer's disease, Huntington's disease, Charcot-Marie-Tooth syndrome, Zellweger syndrome, autoimmune polyglandular syndrome, Marfan's syndrome, Werner syndrome, adrenoleukodystrophy, Menkes syndrome, malignant infantile osteopetrosis, spinocerebellar ataxia, spinal muscular atrophy, or glucose galactose malabsorption. The mesenchymal stem cells may be administered, for example, intravenously or intraosseously. The mesenchymal stem cells may be administered in an amount of from about 1×10⁶ MSCs per kg of body weight to about 10×10⁶ MSCs per kg of body weight. The mesenchymal stem cells may be administered in an amount of from about 4×10⁶ MSCs per kg of body weight to about 8×10⁶ MSCs per kg of body weight.

The technology now will be described with respect to the following examples; however, the scope of the present technology is not intended to be limited thereby.

### EXAMPLE 1

### Mesenchymal Stem Cells for Treatment of Cystic Fibrosis

Increased donor MSC persistence can be achieved by reducing the host MSC population through the use of full body irradiation and/or chemoablative or nonablative procedures before donor MSC delivery to the patient. This procedure provides an open niche for donor MSC engraftment (tissue integration) and has been shown previously to increase MSC migration to the bone marrow. In addition to MSC infusion, delivery of bone marrow cells also will be required to reconstruct the patient's hematopoietic system, which may be destroyed by the methods used to reduce the number of host MSCs in the patient's bone marrow.

MSCs may be delivered by either intravenous infusion or injection directly to the bone marrow cavity (intraosseous injection). Although intravenous MSC delivery may be sufficient for successful MSC integration within the bone marrow of the recipient, intraosseous injection may enhance MSC engraftment persistence. Rapid donor MSC engraftment should increase the likelihood that the exogenously-derived population will be well established before the expansion of any native MSCs that remain after ablative procedures.

A rat model of bone marrow transplant following irradiation is being used to test the hypothesis that either intravenous (IV) or intraosseous (TO) MSC delivery, concurrently with a bone marrow transplant, will result in engraftment following ablative procedures. The protocol also was designed to gain a preliminary comparative measure of the relative success of the two MSC delivery procedures.

On day 0, twelve male Lewis rats were irradiated with 2 fractions of 5.0 Grays (Gy). The radiation fractions were separated by 4 hours. On the following day, bone marrow cells (BMCs) were prepared from an additional 8-10 male Fisher rats. For injection, a total of 30×10⁶ BMCs and 1×10⁶ MSCs in a total volume of 150 ul was used. The MSCs used in the procedure carried the genetic marker human placental alkaline phosphatase (hPAP) for later detection. The experimental design for this study is shown in Table 1 below.

TABLE 1
Study Design. Allocation by experimental group.

| | Number of | | Total Body | |
|---|---|---|---|---|
| | Recipient | | Irradiation | |
| Group | Mice | Treatment | Day −1 | BMT Day 0 |
| 1 | 4 male | Control | 10 Gy* | none |
| | Lewis rats | (no injection) | | |
| 2 | 4 male | Tibial Injection | 10 Gy* | 30 × 10⁶ |
| | Lewis rats | (marrow + | | BM cells |
| | | hPAP cells) | | 1 × 10⁶ hPAP |
| | | | | MSCs |
| 3 | 4 male | IV infusion | 10 Gy* | 30 × 10⁶ |
| | Lewis rats | (marrow + | | BM cells |
| | | hPAP cells) | | 1 × 10⁶ hPAP |
| | | | | MSCs |

*Radiation was divided into 2 fractions of 5.0 Gy. Radiation fractions were separated by 4 hours.

Animals in group 1 (control) received radiation only. Animals in group 2 were injected with MSCs and bone marrow cells directly into the head of the left tibia through the patellar ligament. Animals in group 3 were injected with MSCs and bone marrow cells intravenously.

The animals were weighed and observed daily, and any animal showing obvious signs of pain, such as head bobbing and/or writhing, was treated with buprenorphine. Buprenorphine was administered at a concentration of 0.5 mg/kg (of food) in 6 ml of soft daily food. This treatment started when the animals had lost 15% of their body weight and continued until either scheduled euthanasia or weight loss of greater than 30%. Those animals that were unable to right, cold to the touch, or moribund were euthanized.

The animals were weighed and observed for 14 days. On day 14 all animals were sacrificed and bone marrow was collected from each tibia. The marrow samples were collected into tubes, sealed and packed in ice until they were plated out for assaying.

Bone marrow from each sample then was plated out for the colony forming unit assay. Plates are stained for expression of the hPAP gene. The percentage of exogenously-derived MSCs is determined after counting the number of colonies comprised of stained (exogenously derived) vs. unstained (recipient-derived) MSCs. The resulting data provide an initial assessment as to whether IV or IO delivery is more efficient in establishing the engraftment of donor-derived cells.

Follow-up studies are carried out in a similar manner, but involve experimental subjects that are sacrificed at later time points post-transplantation. In this manner, the persistence of MSC engraftment is determined. The method of MSC delivery for these later experiments will be determined by pilot studies similar to that described above.

Once the procedures for achieving persistent MSC engraftment have been developed in the rat model described above, a rat model of fibrotic lung injury is developed. Rats that have received an MSC transplant are given localized irradiation to the lungs. At various time points post irradiation, animals are sacrificed and the lungs are analyzed for the presence of MSCs by PCR or immunohistochemistry. Significant migration of MSCs to the lungs following radiation injury suggests that MSCs may participate in the healing of various other types of fibrotic lung injury as well.

The underlying cause of fibrotic lung injury in patients who suffer from cystic fibrosis is a genetic defect. If MSCs are obtained from a genetically normal individual and transplanted to cystic fibrosis patients, then the migration of transplanted cells to the lungs in response to the inflammatory signals associated with fibrotic injury would result in an inhibition of the progression of the disease symptoms, or possibly even a reversal of clinical signs. The degree of improvement would be determined by the level of replacement of tissue lining the lungs. The rat model described above in which experimental subjects with traceable MSCs are given localized radiation to the lungs is a surrogate for the fibrotic lung injury that occurs in cystic fibrosis.

Depending upon the outcome of the intravenous and intraosseous administrations of MSCs to rats as hereinabove described in Example 1, a patient with cystic fibrosis is given an intravenous infusion or an intraosseous injection of MSCs (2.5×10⁶ cells/ml) in PlasmaLyteA saline solution (Baxter) to which has been added DMSO at 3.75% vol./vol. and human serum albumin at 1.875% wt./vol. The infusion is continued until the patient receives up to 8 million MSCs per kilogram of body weight. Subsequent infusions of up to 8 million MSCs per kilogram of body weight are given if needed.

The treatment regimen is repeated at one month intervals. Lung function is assessed by spirometry. Treatment is continued until no further improvement in clinical symptoms is observed.

### EXAMPLE 2

### Mesenchymal Stem Cells for Treatment of Wilson's Disease

Depending upon the outcome of the intravenous and intraosseous administrations of MSCs to rats as hereinabove described in Example 1, a patient with Wilson's disease is given an intravenous infusion or an intraosseous injection of MSCs (2.5×10⁶ cells/ml) in PlasmaLyteA saline solution (Baxter) to which has been added DMSO at 3.75% vol./vol. and human serum albumin at 1.875% wt./vol. The infusion is continued until the patient receives up to 8 million MSCs per kilogram of body weight. Subsequent infusions of up to 8 million MSCs per kilogram of body weight are given if needed.

The treatment regimen is repeated at one month intervals. Clinical symptoms are monitored by measuring serum ceruloplasmin, copper levels in the blood and urine, and imaging of the liver (i.e., abdominal X-ray or MRI). Treatment is continued until no further improvement in clinical symptoms is observed.

### EXAMPLE 3

### Mesenchymal Stem Cells for Treatment of Amyotrophic Lateral Sclerosis (ALS)

Depending upon the outcome of the intravenous and intraosseous administrations of MSCs to rats as hereinabove described in Example 1, a patient with ALS is given an intravenous infusion or an intraosseous injection of MSCs (2.5×10⁶ cells/ml) in PlasmaLyteA saline solution (Baxter) to which has been added DMSO at 3.75% vol./vol. and human serum albumin at 1.875% wt./vol. The infusion is continued until the patient receives up to 8 million MSCs per kilogram of body weight. Subsequent infusions of up to 8 million MSCs per kilogram of body weight are given if needed.

The treatment regimen is repeated at one month intervals. Clinical symptoms are monitored by neurological tests, electromyogram (EMG) to test muscle activity, and nerve conduction velocity (NCV) tests to evaluate nerve function. Treatment is continued until no further improvement in motor function is observed.

The disclosures of all patents, publications, including published patent applications, depository accession numbers, and database accession numbers are hereby incorporated by reference to the same extent as if each patent, publication, depository accession number, and database accession number were specifically and individually incorporated by reference.

It is to be understood, however, that the scope of the present technology is not to be limited to the specific embodiments described above. The technology may be practiced other than as particularly described and still be within the scope of the accompanying claims.

1. A method of treating cystic fibrosis comprising:
administering to a human patient with cystic fibrosis allogeneic human mesenchymal stem cells in an amount effective to treat cystic fibrosis in said patient.

2. The method of claim 1, wherein said mesenchymal stem cells are administered intravenously.

3. The method of claim 1, wherein said mesenchymal stem cells are administered intraosseously.

4. The method of claim 1, wherein said mesenchymal stem cells are administered in a solution comprising dimethyl sulfoxide and human serum albumin.

5. The method of claim 1, wherein said mesenchymal stem cells are administered in an amount of from about 1×10⁶ cells per kilogram of body weight to about 10×10⁶ cells per kilogram of body weight.

6. The method of claim 1, wherein said mesenchymal stem cells are administered in an amount of from about 4×10⁶ cells per kilogram of body weight to about 8×10⁶ cells per kilogram of body weight.

7. The method of claim 1, wherein said mesenchymal stem cells are administered as a suspension of cells.

8. The method of claim 7, wherein at least about 95% of the cells in said suspension express one or more cell surface markers selected from the group consisting of CD73, CD105, and CD166.

9. The method of claim 7, wherein at least about 98% of the cells in said suspension express one or more cell surface markers selected from the group consisting of CD73, CD105, and CD166.

10. A method of treating cystic fibrosis in a human patient comprising:
reducing an endogenous mesenchymal stem cell population of said human patient with cystic fibrosis; and
administering to said patient a human cell suspension in an amount effective to treat cystic fibrosis in said patient,
wherein at least about 95% of the cells in said suspension express one or more cell surface markers selected from the group consisting of CD73, CD105, and CD166.

11. The method of claim 10, wherein at least about 98% of the cells in said suspension express one or more cell surface markers selected from the group consisting of CD73, CD105, and CD166.

12. The method of claim 10, wherein said cell suspension is administered intravenously.

13. The method of claim 10, wherein said cell suspension is administered intraosseously.

14. The method of claim 10, wherein said suspension comprises dimethyl sulfoxide and human serum albumin.

15. The method of claim 10, wherein said cell suspension comprises from about 1×10⁶ cells per kilogram of body weight to about 10×10⁶ cells per kilogram of body weight.

16. The method of claim 10, wherein said cell suspension comprises from about 4×10⁶ cells per kilogram of body weight to about 8×10⁶ cells per kilogram of body weight.

17. A method of delivering a normal CFTR gene to the lung of a human cystic fibrosis patient comprising:
systemically administering to said patient with cystic fibrosis allogeneic human mesenchymal stem cells, wherein the mesenchymal stem cells migrate to and engraft in the lung.

18. The method of claim 17, wherein said mesenchymal stem cells are administered intravenously.

19. The method of claim 17, wherein said mesenchymal stem cells are administered intraosseously.

20. The method of claim 17, wherein said mesenchymal stem cells are administered in a solution comprising dimethyl sulfoxide and human serum albumin.

21. The method of claim 17, wherein said mesenchymal stem cells are administered in an amount of from about 1×10⁶ cells per kilogram of body weight to about 10×10⁶ cells per kilogram of body weight.

22. The method of claim 17, wherein said mesenchymal stem cells are administered in an amount of from about 4×10⁶ cells per kilogram of body weight to about 8×10⁶ cells per kilogram of body weight.

23. The method of claim 17, wherein said mesenchymal stem cells are administered as a suspension of cells.

24. The method of claim 23, wherein at least about 95% of the cells in said suspension express one or more cell surface markers selected from the group consisting of CD73, CD105, and CD166.

25. The method of claim 24, wherein at least about 98% of the cells in said suspension express one or more cell surface markers selected from the group consisting of CD73, CD105, and CD166.
